(19) United States
(12) Patent Application Publication
(10) Pub. No.: US 2005/0055013 A1
(43) Pub. Date: Mar. 10, 2005

(54) Medication delivery device

(76) Inventor: Anne Marie Chalmers, Osprey, FL (US)

Correspondence Address: LAHIVE & COCKFIELD, LLP., 28 STATE STREET, BOSTON, MA, 02109, US

(21) Appl. No.: 10/690,387

(22) Filed: Oct. 21, 2003

Related U.S. Application Data
(63) Continuation-in-part of application No. 10/657,521, filed on Sep. 8, 2003.

(51) Int. Cl. A61K 9/48; A61K 9/20; A61K 9/22

(52) U.S. Cl. 604/890.1; 424/464

(57) ABSTRACT

A medication delivery device by which multiple medications are encased within separate containers so that each of the said medications are not in actual physical contact with each other until the said containers degrade within a live body, with each of the said separate containers being themselves externally physically connected to at least one other separate container in sequential, chain fashion, which containers are biodegradable within a live body.

### CROSS-REFERENCE TO RELATED APPLICATIONS

This Application is a Continuation-in-Part of application Ser. No. 10/657,521 entitled Medication Delivery Device, filed by the same sole inventor as herein with the United States Patent and Trademark Office on Sep. 8, 2003, which application is pending.

### STATEMENT REGARDING FEDERALLY SPONSORED RESEARCH OR DEVELOPMENT

Not Applicable

### REFERENCE TO SEQUENCE LISTING, A TABLE OR A COMPUTER PROGRAM LISTING COMPACT DISK APPENDIX

Not Applicable

### BACKGROUND OF INVENTION

This invention relates to a device for the simultaneous delivery into a live body of multiple medication products, pharmaceuticals, nutritional products and inert materials.

As used herein, the phrase “pill”, “capsule”, and “softgel” are used interchangeably, unless specifically otherwise limited in scope at a specific section herein, to include and encompass, and not necessarily be limited to “pills and capsules and soft-gels”, and indeed encompass all other mechanisms and means for delivery of medication products into a live body.

As used herein, the phrase “medication product” includes and encompasses, but is not limited to, prescription drugs, non-prescription drugs, over-the-counter drugs, nutritional supplements and inert “filler” materials.

As used herein, the phrases “container” and “containment means” includes and encompasses not only the traditional medication capsule, pill, gel cap, suppositories, skin patches and sublingual applications, but also includes and encompasses any and all other medication delivery mechanisms.

As used herein, the phrase “outer shell” means the container or containment means which itself is not itself connected with or affixed to another or further container or containment device in chain, train or string fashion, which said non-connected and non-affixed container or containment means is intended for ingestion or insertion into a live body.

Due to an increasing aging population and increased use of drug therapy, more and more people find themselves taking several pills, tablets or capsules for treating or preventing illnesses every day. Research has shown that even patients for whom strict adherence to prescribed drug regimens is crucial, rates of non-compliance can still range from as much as 20% to 50%.

Each year in the United States, the consequences of poor compliance cost an estimated $100 billion in added health care expenses, lost productivity, and other direct and indirect costs, in addition to personal suffering.

One means of increasing compliance is to reduce the number of pills taken per day, thus reducing patient resistance to swallowing large numbers of pills or the possibility of patients forgetting to take some of their medication.

Moreover, numerous studies have shown that certain combinations of different substances or medications can dramatically improve the health outcomes through additional or synergic effects. But these combinations most often require the ingestion of more pills which may again lower the compliance.

The delivery of medication products, including prescription drugs, over-the-counter drugs, nutritional supplements and inert materials, has been traditionally accomplished by the use of pills, capsules, and gel caps.

Typically pills are comprised of the active ingredient compounded with inert ingredients for various purposes, including ease of handling small amounts of active ingredients, with that mixture of active and inactive ingredients being then compressed to form a “pill”. Very often medications can come in a dose of only a few milligrams, but since this amount is so small and unmanageable, the size of the pill has to be increased substantially with fillers

Similarly, capsules and gel caps are composed of an outer material or casing which is dissolved after ingestion by the patient, with the interior portion of the capsule or gel cap being filled with an active ingredient compounded with inert ingredients for various purposes, including ease of handling of small amounts of active ingredients, similar to a pill.

There are several distinctions between capsules vis-à-vis soft gels or gel caps. The major difference between a capsule and a soft gel or gel cap is that a capsule is a hard shell and a soft gel or gel cap is physically a softer gelatin container. A soft gel or gel cap is essentially a capsule made from gelatin, usually from a bovine or pig source, although there are also available soft gels made from vegetable sources, e.g., potato starch, whereas a capsule can be generally be made from many different materials, including gelatin formulations. Generally capsules usually contain solid materials such as powders, although they occasionally do contain liquids, whereas soft gels or gel caps usually contain oils or liquid, although some soft gels or gel caps do contain rather powdery substances in soft gels or gel caps, which facilitate the swallowing process.

A sub-category or variation of gel caps is “liquid gel caps”, which are gel caps, the interior of which are filled with liquid rather than solid materials, typically a blend of active and inactive ingredients. The liquid gel caps provide an easy means of carrying liquid medications for ingestion without the need to transport the liquid from a liquid container to the mouth of the patient, thus simultaneously avoiding the risk of liquid bottle spillage or breakage.

The disadvantage of a typical prescription pill, capsule or gel cap is that they usually contain only one primary medication thus only addressing one type of indication or problem.

The treatment of many patients requires the use of multiple medications by the patient, as to which the use of numerous traditional pills, capsules or gel caps is burdensome. Not only does the patient need to be burdened with multiple containers for the various medications, but the patient must also track each pill, capsule or gel cap to assure that they have in fact timely ingested the proper dosage of each such medication.

An objective of the present invention is to solve the aforesaid problems, including by reducing the number of pills that will contain the originally intended, prescribed or recommended medications and doses, thus increasing compliance and reducing the possibility of confusion.

### BRIEF SUMMARY OF THE INVENTION

A medication delivery device by virtue of which multiple medications are encased within separate containers so that each of the said medications are not in actual physical contact with each other until the said containers degrade within a live body, with said separate containers being themselves sequentially connected and affixed to each other in chain type fashion in a series or string of such containers, that is, containers in a string or train of other containers, which containers are biodegradable within a live body.

The several components of the said medication delivery device which is the invention herein may be variously constructed of material which is opaque, translucent or transparent.

One of several preferred embodiments of the invention is a first capsule connected with and affixed to a second capsule, wherein the said first capsule is filled with a first medication, with that first capsule then connected with and affixed to a second capsule, with that second capsule containing a second medication.

### BRIEF DESCRIPTION OF THE DRAWINGS

FIG. 1 is a cross-section of a preferred embodiment of the invention, depicting in this embodiment two separate compartments physically connected with each other.

FIG. 2 is a cross-section of the same embodiment as depicted in FIG. 1 except with the two separate compartments being physically disconnected from each other.

FIG. 3 is a cross-section of a close-up magnification of the interface of two of the several compartments with each other.

FIG. 4 is a cross-section of a close-up magnification of the interface of two of the several compartments connected with and affixed to each other, in which is depicted one embodiment of a means of connecting the two said separate compartments with each other.

FIG. 5 is a cross-section of a close-up magnification of the interface of two of the several compartments connected with and affixed to each other, in which is depicted one alternate embodiment of a means of connecting the two said separate compartments with each other.

FIG. 6 is a cross-section of a close-up magnification of the interface of two of the several compartments connected with and affixed to each other, in which is depicted a further and additional alternate embodiment of a means of connecting the two said separate compartments with each other.

FIG. 7 is a cross-section of a preferred embodiment of the invention, depicting in this embodiment three separate compartments physically connected with each other.

FIG. 8 is a cross-section of the same embodiment as depicted in FIG. 7 except with the three separate compartments being physically disconnected from each other.

FIG. 9 is a cross section of another embodiment of the invention, depicting two separate compartments physically connected with each other.

FIG. 10 is a cross section of a further embodiment of the invention, depicting two separate compartments physically connected with each other.

FIG. 11 is a cross section of yet another embodiment of the invention, depicting two separate compartments physically connected with each other.

FIG. 12 is a cross section of a yet further embodiment of the invention, depicting two separate compartments physically connected with each other.

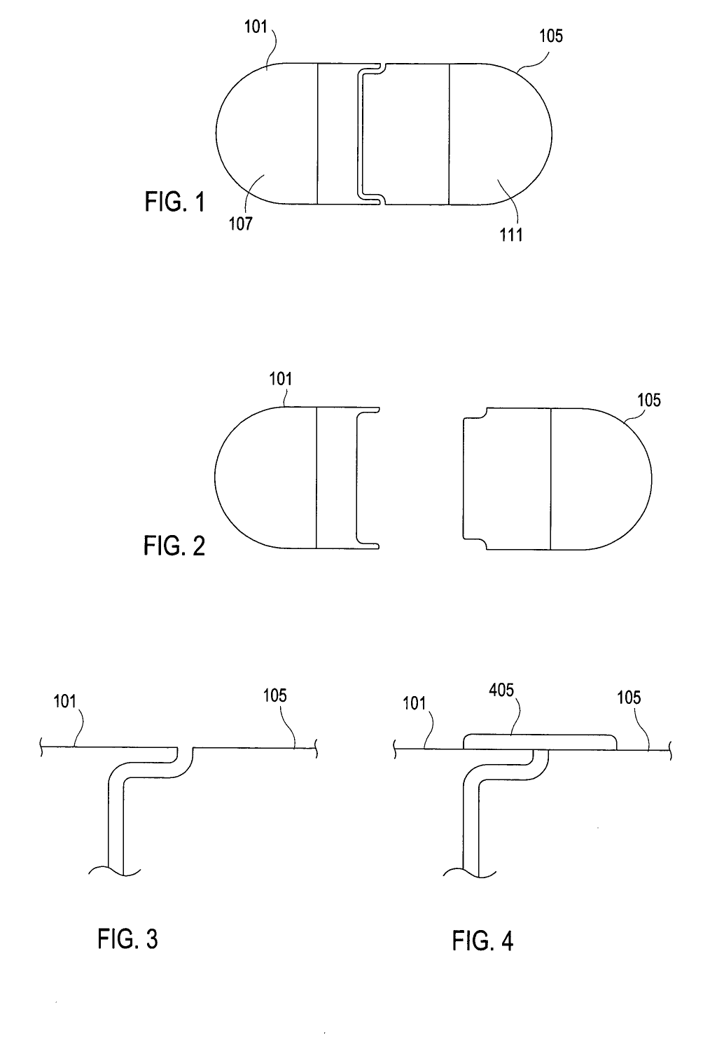

These FIGS. 1 through FIG. 12 are not necessarily exhaustive of all embodiments of the invention.

### DETAILED DESCRIPTION OF THE INVENTION

A medication delivery device in accordance, as exemplified by the several preferred embodiments depicted in FIGS. 1 through 12, provides for the delivery to a living body, human or animal, of multiple medication products simultaneously.

Although the more typical method and means for entry into the said living body is by ingestion, the invention also encompasses other forms of such entry, including but not limited to suppository form or application to the skin.

As depicted in FIG. 1, one preferred embodiment of the invention is comprised of a first capsule (101) connected with and affixed to a second capsule (105), and with the first capsule (101) filled with a first medication (107), and the second capsule (105) filled with a second medication (111).

The first medication (107) and the second medication (111) are pharmacologically distinct from each other.

The said first medication (107) and the said second medication (111) may function in a synergistically physiological manner in conjunction with either or both or none of the other said medications.

For example, the said first medication (107) could be a prescription pharmaceutical or a non-prescription nutritional supplement, and the said second medication (111) could be a prescription pharmaceutical or a non-prescription nutritional supplement, with the said medications (107) and (111) having clinically established the efficacy of enhancing each other.

Alternatively, there need not be any interaction between the said first medication (107) and the said second medication (111). For example, each said medication can have separate and different effects on the body.

FIG. 2 depicts the capsule of FIG. 1, but with the separate compartments or segments (101) and (105) being physically separate from each other, demonstrating that each said segment or component or compartment is completely separate from the other until such time that they are assembled, connected with and affixed to each other.

FIG. 3 depicts the interface of the two separate compartments (101) and (105) with each other in one embodiment of the invention, showing particularly the mating of the points of contact of capsules (101) and (105) with each other.

FIG. 4 is an embellishment of FIG. 3, with FIG. 4 depicting one embodiment of the invention in which the said capsules (101) and (105) are attached with and affixed to each other by means of a band of material (405) overlapping both capsules (101) and (105) along the length of the perimeter of their respective circumferences at the interface of capsules (101) and (105) with each other.

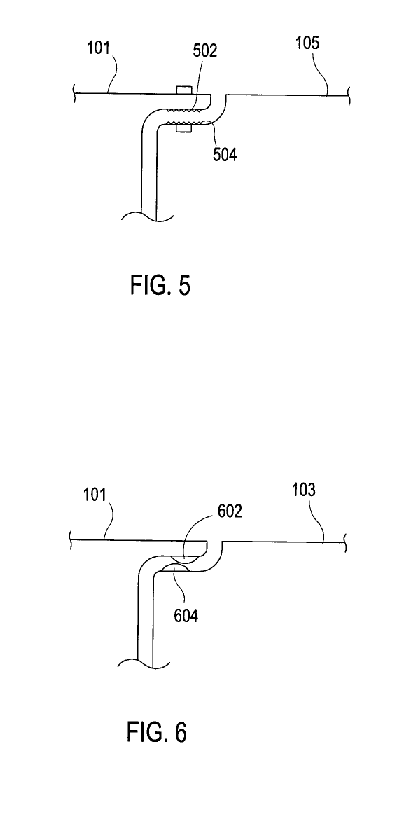

FIG. 5 is an alternate embellishment of FIG. 3, with FIG. 5 depicting one embodiment of the invention in which the said capsules (101) and (105) are attached with and affixed to each other by means of screw-type threads (502) and (504) respectively, which mate with each other as capsules (101) and (105) are rotated and inserted into each other as a means of connecting and affixing capsules (101) and (105) with each other.

FIG. 6 is a further alternate embellishment of FIG. 3, with FIG. 6 depicting one embodiment of the invention in which the said capsules (101) and (105) are attached with and affixed to each other by means of snap interlocking means (602) and (604) respectively, in which the respective nodules (602) and (604) are mechanically plastically deformable, thereby allowing and enabling nodule (602) on capsule (101) to slid and slip over nodule (604) in capsule (105), resulting in the then connected and affixed assemblage of capsule (101) with and to capsule (105) being separable from each other only upon the exertion of substantial force applied to capsule (101) and to capsule (105) simultaneously and in directions opposite to each other.

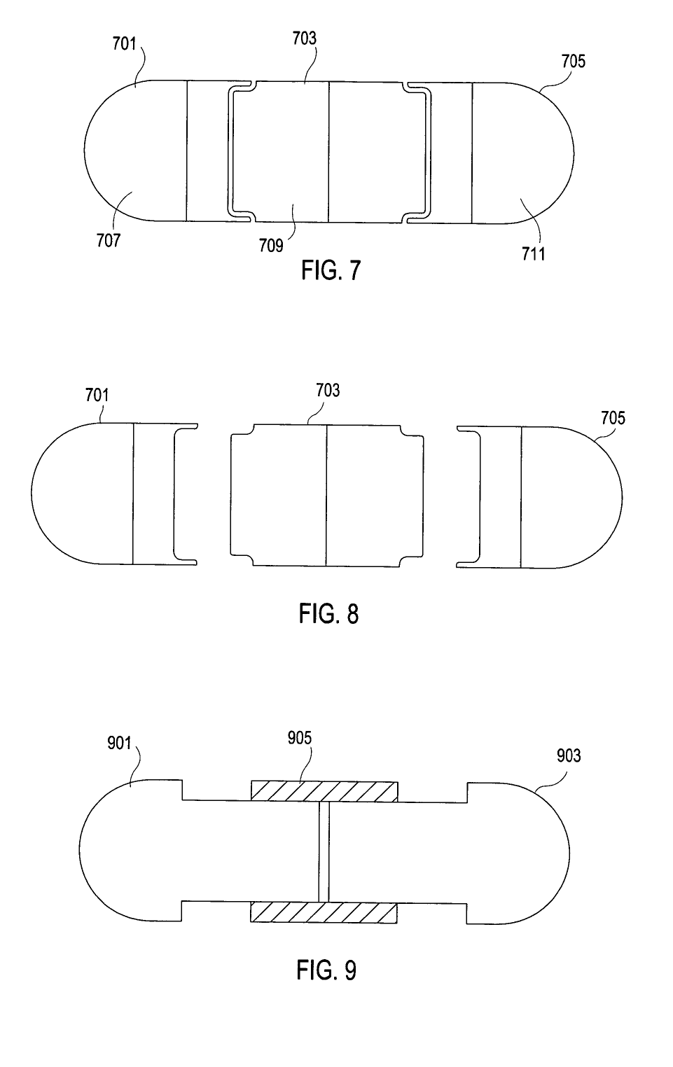

As depicted in FIG. 7, a further embodiment of the invention is comprised of a capsule (701) connected with and affixed to a second capsule (703), with that second capsule (703) then connected with and affixed to a third capsule (705), with the first capsule (701) and the third capsule (705) not directly connected to each other, and with the first capsule (701) filled with a first medication (707), and the second capsule (703) filled with a second medication (709), and the third capsule (705) filled with a third medication (711).

The said first medication (707), the said second medication (709) and the said third medication (711) are pharmacologically distinct from each other.

The said first medication (707), the said second medication (709) and the said third medication (711) may function in a synergistically physiological manner in conjunction with either or both or none of the other said medications.

For example, the first medication (707) could be a prescription pharmaceutical and the said second medication (709) could be a non-prescription nutritional supplement, and the said third medication (711) could be either a prescription pharmaceutical or a non-prescription nutritional supplement, with the said medications (707), (709) and (711) having clinically established the efficacy of enhancing the other said medications.

Alternatively, there need not be any interaction between the first medication and/or the second medication and/or the third medication. For example each medication can have separate and different effects on the body.

FIG. 8 depicts the capsule of FIG. 7, but with the separate compartments or segments physically separate from each other, demonstrating that each segment or component or compartment is completely separate from the others until such time that they are assembled, connected with and affixed to other components or segments.

FIG. 9 is a further embodiment of the invention with three capsules (901) and (903) instead of two, and with the lateral surface of both (901) and (903) respectively being relatively indented, that is, having a shorter circumference from the longitudinal center axis of the center line of each of said capsules, than of (901) and (903) at the closed extremities of the said capsules, and with said capsules (901) and (903) being attached with and affixed to each other by means of a band of material (905) overlapping both capsules (701) and (703) along the length of the perimeter of their respective circumferences, with said band (905) being of such thickness that it fits into the said indented lateral surface of both (901) and (903), resulting in the assemblage of capsule (901) and (903) with band (905) attached having a relatively smooth surface along the entire lateral surface of those thus assembled capsules.

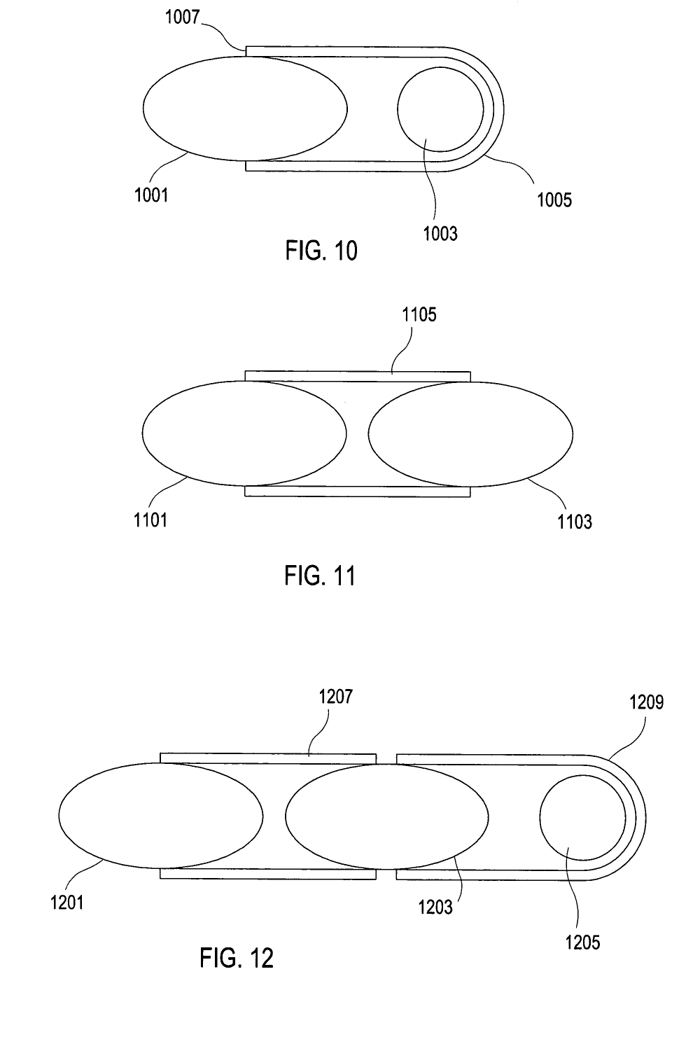

FIG. 10 is a further embodiment of the invention in which is depicted one large pill (1001) assembled in association with a smaller pill (1003), with said smaller pill (1003) being inserted into a cup type device means (1005), with said cup type device means (1005) then connected with and affixed to the larger pill (1001) at connection area (1007) by various means, including but not limited to a pressure fit between the outside surface of larger pill (1001) in contact with the inner surface of the cup type device means (1005).

FIG. 11 is a further embodiment of the invention in which is depicted a first gel cap (1101) assembled in association with a second gel cap (1103), with both of said gel caps (1101) and (1103) being connected with and affixed to each other by means of a band of material (1105) around the mid-section of each of the said gel caps (1101) and (1103).

FIG. 12 is a further embodiment of the invention in which is depicted a first pill (1201) assembled in association with and physically connected to a second pill (1203) by means of a band of material (1207) surrounding the one-half of the said first pill (1201) and the one-half of the said second pill (1203) which are in closest proximity to each other, as depicted in FIG. 11, with the said assemblage of the said first pill (1201) and the said second pill (1203) then further assembled in association with and is connected with a third pill (1205), which said third pill (1205) is inserted or otherwise contained within a cup type device means (1209), with said cup type device means (1209) then connected and with the said assemblage of pills (1201) and (1203) connected and affixed to each other with band (1207) then being subsequently connected with and affixed to the said cup type device means (1209), at a connection area by various means, including but not limited to a pressure fit between the outside surface of second pill (1203) and the inner surfaces of band (1207) and (1209).

Although the foregoing embodiments refer to capsules, the invention is not limited to capsules, but rather encompasses any and all medication containers and containment means, including capsules, but also including but not limited to gel caps and pills as discussed herein.

In addition, although several of the preferred embodiments described hereinbefore are comprised, for illustration purposes, of only one or two capsules, gel caps or pills, or combinations thereof, the invention is not limited to a specific number of capsules, pills and/or gel caps, but rather encompasses any number of containers or containment means.

A very small list of numerous examples of types and categories of medication products which would benefit from the use of this invention include: (a) statin+aspirin; (b) Statin+aspirin+omega-3; and (c) Statin+aspirin+omega 3+a blood pressure medication; and (d) fat soluble essential element+water soluble element (such as omega 3+vitamin B complex).

Each container, segment and compartment is separated from the other ones by being a distinct and free-standing unit, preventing the medication substance in one container compartment from mixing with the medication substance in the other container compartments.

Consequently, there is no concern about the medication substances starting to react with each other within the most outer container compartment, and the medications substances will therefore not represent a new chemical compound, before being ingested or otherwise delivered into a live body.

Furthermore, by virtue of the container compartments being “separate chambers”, the highly economically attractive and flexible prospect of different medication substances actually being produced at locations distant from each other and then being consolidated in medication delivery devices in accordance with this invention become feasible, and indeed economically attractive.

The numerous advantages of this invention include: (a) The ability to maintain the chemical stability of the different medication substances, by preventing any chemical reaction between or among them by virtue of the fact that they are separated from each other by being in separate containers and compartments; (b) ease of production of a means to deliver multiple medications simultaneously, by virtue of each container compartment being capable of being produced and filled with different medications at remote distant locations, before the final assemblage; (c) flexibility, since the content or concentration can be changed for one substance without influencing the chemical properties of the other; (d) increased intestinal absorption, since the bio-availability is usually higher for soft gels and capsules compared to tablets or pills; (e) increased patient compliance and assurance that the patient is actually taking several medical substances since they come as “one dose”; (f) increased patient compliance because the patient will be more willing to take one pill compared to several; (g) high patient compliance because patients often have an easier time swallowing a capsule or soft gel or gel cap compared to a tablet or pill; (h) a precise “medical” communication, because it shows clearly which products are combined (not mixed), and thus opening the area of “synergy” medicine, which is especially evident when the material band (1105) and (1207) or the cup type device means (1005) and (1209), or indeed any component of the said medication delivery device of this invention, is translucent or transparent rather than opaque, thus allowing the contents therein to be readily viewed externally; and (i) by using a translucent or transparent rather than opaque material band (1105) and (1207) or cup type device means (1005) and (1209) to affix, connect or contain more than one medication product, pill, capsule or gel cap to increase the quality assurance since the said internally contained medicinal products can have different colors or shapes related to the identity or dosage, thus allowing for inspection to assure and confirm that the medication delivery device does in fact actually contain the appropriate medication product.

It is contemplated that the inventive concepts herein described may be variously otherwise embodied and it is intended that the appended claims be construed to include alternative embodiments of the invention except only insofar as limited by prior art.

Furthermore, by standardizing the diameter and the shape of the different containers, it becomes possible to thereby obtain an optimal physical configuration that favors the swallowing reflex of a live body and also reduces the “dead” space to a minimum, thus making the total volume of the medicinal delivery device as small and as compacted as possible.

In addition, by standardizing the diameter and the shapes of the different containers, it becomes possible to customize the medicinal product delivery device to the individual patient, with the ability to interchange the different components to adjust for different medications or dosages.

Also by standardizing the diameter and the shape of the different containers, we allow for putting many medications into “one” pill, capsule or gel cap, rather than the otherwise need to use several.

Another advantage of the invention is that by standardizing the diameter and the shape of the different containers, it becomes possible to build automated feeding machines at an assembly line that can easily put the desired components together coming from different feeders depending on the programmed information, which could be a medical prescription coming from a physician.

1. A device for the simultaneous delivery of multiple medication products to a live body, with such device comprising:
a first dosage of a first medication product contained within a first containment means;
a second dosage of a second medication product contained within a second containment means; and
the first containment means affixed by affixing means to the second containment means.

2. A device for the simultaneous delivery of multiple medication products to a live body, with such device comprising:
a first dosage of a first prescription pharmaceutical product or non-prescription nutritional supplement product contained within a first pill, capsule or gel cap;
a second dosage of a second prescription pharmaceutical product or non-prescription nutritional supplement product contained within a second pill, capsule or gel cap; and
the first pill, capsule or gel cap affixed by affixing means to the second pill, capsule or gel cap.

3. A device for the simultaneous delivery of multiple medication products to a live body, with such device comprising:
a first dosage of a first medication product contained within a first containment means;
a second dosage of a second medication product contained within a second containment means; and
the first containment means affixed by affixing means externally to the second containment means, with the first containment means and the second containment means so mutually affixed to each other to thereby constitute one containment means.

4. A device for the simultaneous delivery of multiple medication products to a live body, with such device comprising:
a first dosage of a first prescription pharmaceutical product or non-prescription nutritional supplement product contained within a first pill, capsule or gel cap;
a second dosage of a second prescription pharmaceutical product or non-prescription nutritional supplement product contained within a second pill, capsule or gel cap; and
the first pill, capsule or gel cap affixed by affixing means to the second pill, capsule or gel cap to constitute one capsule or gel cap.

5. A device for the simultaneous delivery of multiple medication products to a live body, with such device comprising:
a first dosage of a first medication product contained within a first containment means;
a second dosage of a second medication product contained within a second containment means; and
the first containment means affixed by affixing means externally to the second containment means, with the first containment means and the second containment means so mutually affixed to each other to thereby constitute one elongated containment means.

6. A device for the simultaneous delivery of multiple medication products to a live body, with such device comprising:
a first dosage of a first prescription pharmaceutical product or non-prescription nutritional supplement product contained within a first pill, capsule or gel cap;
a second dosage of a second prescription pharmaceutical product or non-prescription nutritional supplement product contained within a second pill, capsule or gel cap; and
the first pill, capsule or gel cap affixed by affixing means to the second pill, capsule or gel cap to constitute one elongated capsule or gel cap.

7. A device for the simultaneous delivery of multiple medication products to a live body, with such device comprising:
a first dosage of a first medication product contained within a first containment means;
a second dosage of a second medication product contained within a second containment means;
a third dosage of a third medication product contained within a third containment means; and
the said first containment means affixed by affixing means to the said second containment means and the said second containment means affixed to the said third containment means, but with the said first containment means not directly affixed to the said third containment means.

8. A device for the simultaneous delivery of multiple medication products to a live body, with such device comprising:
a first dosage of a first prescription pharmaceutical product or non-prescription nutritional supplement product contained within a first pill, capsule or gel cap;
a second dosage of a second prescription pharmaceutical product or non-prescription nutritional supplement product contained within a second pill, capsule or gel cap;
a third dosage of a third prescription pharmaceutical product or non-prescription nutritional supplement product contained within a third pill, capsule or gel cap;
the said first pill, capsule or gel cap affixed by affixing means to the said second pill, capsule or gel cap and the said second pill, capsule or gel cap affixed to the said third pill, capsule or gel cap, but with the said first pill, capsule or gel cap not directly affixed to the said third pill, capsule or gel cap.

9. A device for the simultaneous delivery of multiple medication products to a live body, with such device comprising:
a first dosage of a first medication product contained within a first containment means;
a second dosage of a second medication product contained within a second containment means;
a third dosage of a third medication product contained within a third containment means; and
the said first containment means affixed by affixing means to the said second containment means and the said second containment means affixed to the said third containment means, but with the said first containment means not directly affixed to the said third containment means; and
the said first containment means so affixed by affixing means to the said second containment means, which is so affixed to the said third containment means such to thereby constitute one containment means.

10. A device for the simultaneous delivery of multiple medication products to a live body, with such device comprising:
a first dosage of a first prescription pharmaceutical product or non-prescription nutritional supplement product contained within a first pill, capsule or gel cap;
a second dosage of a second prescription pharmaceutical product or non-prescription nutritional supplement product contained within a second pill, capsule or gel cap;
a third dosage of a third prescription pharmaceutical product or non-prescription nutritional supplement product contained within a third pill, capsule or gel cap;
the said first pill, capsule or gel cap affixed by affixing means to the said second pill, capsule or gel cap and the said second pill, capsule or gel cap affixed by affixing means to the said third pill, capsule or gel cap, but with the said first pill, capsule or gel cap not directly affixed to the said third pill, capsule or gel cap to thereby constitute one capsule or gel cap.

11. A device for the simultaneous delivery of multiple medication products to a live body, with such device comprising:
a first dosage of a first medication product contained within a first containment means;
a second dosage of a second medication product contained within a second containment means;
a third dosage of a third medication product contained within a third containment means;
said first containment means affixed by affixing means to the said second containment means and the said second containment means affixed by affixing means to the said third containment means, but with the said first containment means not directly affixed to the said third containment means; and
said first containment means so affixed by affixing means to the said second containment means, which is so affixed by affixing means to the said third containment means such to thereby constitute one elongated containment means.

12. A device for the simultaneous delivery of multiple medication products to a live body, with such device comprising:
a first dosage of a first prescription pharmaceutical product or non-prescription nutritional supplement product contained within a first pill, capsule or gel cap;
a second dosage of a second prescription pharmaceutical product or non-prescription nutritional supplement product contained within a second pill, capsule or gel cap;
a third dosage of a third prescription pharmaceutical product or non-prescription nutritional supplement product contained within a third pill, capsule or gel cap;
the said first pill, capsule or gel cap affixed by affixing means to the said second pill, capsule or gel cap and the said second pill, capsule or gel cap affixed by affixing means to the said third pill, capsule or gel cap, but with the said first pill, capsule or gel cap not directly affixed to the said third pill, capsule or gel cap to thereby constitute one elongated capsule or gel cap.

13. The device of claim 1 in which the said affixing means is transparent.

14. The device of claim 2 in which the said affixing means is transparent.

15. The device of claim 3 in which the said affixing means is transparent.

16. The device of claim 4 in which the said affixing means is transparent.

17. The device of claim 5 in which the said affixing means is transparent.

18. The device of claim 6 in which the said affixing means is transparent.

19. The device of claim 7 in which the said affixing means is transparent.

20. The device of claim 8 in which the said affixing means is transparent.

21. The device of claim 9 in which the said affixing means is transparent.

22. The device of claim 10 in which the said affixing means is transparent.

23. The device of claim 11 in which the said affixing means is transparent.

24. The device of claim 12 in which the said affixing means is transparent.

25. The device of claim 1 in which the said affixing means is translucent.

26. The device of claim 2 in which the said affixing means is translucent.

27. The device of claim 3 in which the said affixing means is translucent.

28. The device of claim 4 in which the said affixing means is translucent.

29. The device of claim 5 in which the said affixing means is translucent.

30. The device of claim 6 in which the said affixing means is translucent.

31. The device of claim 7 in which the said affixing means is translucent.

32. The device of claim 8 in which the said affixing means is translucent.

33. The device of claim 9 in which the said affixing means is translucent.

34. The device of claim 10 in which the said affixing means is translucent.

35. The device of claim 11 in which the said affixing means is translucent.

36. The device of claim 12 in which the said affixing means is translucent.

37. The device of claim 3 in which the said one containment means is transparent.

38. The device of claim 3 in which the said one containment means is translucent.

39. The device of claim 4 in which the said one capsule or gel cap is transparent.

40. The device of claim 4 in which the said one capsule or gel cap is translucent.

41. The device of claim 5 in which the said one elongated containment means is transparent.

42. The device of claim 5 in which the said one elongated containment means is translucent.

43. The device of claim 6 in which the said one elongated capsule or gel cap is transparent.

44. The device of claim 6 in which the said one elongated capsule or gel cap is translucent.

45. The device of claim 9 in which the said one containment means is transparent.

46. The device of claim 9 in which the said one containment means is translucent.

47. The device of claim 9 in which the said one capsule or gel cap is transparent.

48. The device of claim 9 in which the said one capsule or gel cap is translucent.

49. The device of claim 10 in which the said one capsule or gel cap is transparent.

50. The device of claim 10 in which the said one capsule or gel cap is translucent.

51. The device of claim 11 in which the said one elongated containment means is transparent.

52. The device of claim 11 in which the said one elongated containment means is translucent.

53. The device of claim 12 in which the said one elongated capsule or gel cap is transparent.

54. The device of claim 12 in which the said one elongated capsule or gel cap is translucent.
